(19) United States
(12) Patent Application Publication
(10) Pub. No.: US 2012/0173287 A1
(43) Pub. Date: Jul. 5, 2012

(54) Medical Compliance Kiosk

(76) Inventor: Nelson Parker Cowand, Gorham, ME (US)

(21) Appl. No.: 13/392,355

(22) PCT Filed: Aug. 31, 2010
(86) PCT No.: PCT/US10/47410

§ 371 Date: Feb. 24, 2012

Related U.S. Application Data
(60) Provisional application No. 61/275,545, filed on Aug. 31, 2009.
(60) Provisional application No. 61/260,036, filed on Nov. 11, 2009.
(60) Provisional application No. 61/300,965, filed on Feb. 3, 2010.
(60) Provisional application No. 61/374,410, filed on Aug. 17, 2010.

(51) Int. Cl. G06Q 50/22

(52) U.S. Cl. 705/3

(57) ABSTRACT

A medical compliance kiosk (MCK) is an electronic device configured to help a healthcare service provider (HSP) track drug samples, equipment, inventory, medical records, and other valuable information, including information related to regulatory compliance. MCK may also provide electronic medical records and electronic prescription services. The kiosk is a dedicated hardware device with a touch-sensitive input screen. MCK provides a streamlined procedure for tracking critical information and maintaining records.

### CROSS REFERENCE TO RELATED APPLICATIONS

This application is a national stage entry of PCT Application PCT/US10/47410, titled “Medical Compliance Kiosk,” filed Aug. 31, 2010, which claims priority to the following U.S. provisional applications: 61/275,545, entitled “Medical Integrity and Compliance Kiosk,” filed Aug. 31, 2009; 61/260,036, entitled “Medical Regulation Compliance System,” filed Nov. 11, 2009; 61/300,965, entitled “Medical Information Compliance Kiosk,” filed Feb. 3, 2010; and 61, 374,410, entitled “Medical Compliance Kiosk,” filed Aug. 17, 2010. All of the foregoing are incorporated herein by reference.

### BACKGROUND

This specification relates to the field of health care services and more particularly to a medical compliance kiosk (MCK) configured to interface with a Medical Practice Information Network (MPIN).

A physician or other health care service provider (HSP) practices in a heavily-regulated industry. Compliance with health care regulations may impose a substantial burden on the HSP.

### BRIEF DESCRIPTION OF THE DRAWINGS

FIG. 1 is a block diagram of a Medical Practice Information Network.

FIG. 2 is a front view of an exemplary embodiment of a Medical Compliance Kiosk.

FIG. 3 is a block diagram of internal hardware for a Medical Compliance Kiosk.

FIG. 4 is an exemplary user interface screen for a physician viewing a patient's drug history.

FIG. 5 is an exemplary user interface screen for a patient to select physicians allowed to view his or her medication history.

FIG. 6 is an exemplary user interface screen for a patient to select medications that a particular physician is permitted to see.

FIG. 7 is an exemplary user interface screen for a patient or a physician to add a new medication to the patient's medication database.

### SUMMARY OF THE INVENTION

In one aspect, a medical compliance kiosk (MCK) is an electronic device configured to help a healthcare service provider (HSP) track drug samples, equipment, inventory, medical records, and other valuable information, including information related to regulatory compliance. MCK may also provide electronic medical records and electronic prescription services. The kiosk is a dedicated hardware device with a touch-sensitive input screen. MCK provides a streamlined procedure for tracking critical information and maintaining records.

### DETAILED DESCRIPTION OF THE EMBODIMENTS

A medical compliance kiosk (MCK) may be provided as part of a larger medical practice information network (MPIN). At the core of the MPIN is a central server, which is configured to receive data from a plurality of MCKs and to store the data in a central database. Because data are stored in a central database instead of on the local MCK, sensitive information is not subject to malicious or accidental disclosure from the local MCK, and a patient who sees several HSPs can maintain a consistent electronic medical record (EMR).

In the exemplary embodiment, MCK is a fully integrated with the MPIN to monitor numerous aspects of a medical practice. Although MCK is spoken of as a “kiosk,” the exemplary embodiment includes a plurality of computing devices connected to each other on an internal network, and also configured to connect to an external network such as the Internet. Persons having ordinary skill in the art will appreciate that although this specification assigns certain functions to particular nodes in this network, many functions can be performed at more than one node. For example, the exemplary embodiment disclosed in this specification includes the feature that local nodes located on-site do not store database information, but rather transmit information to a central server where it can be stored in a central database. But those having skill in the art will appreciate that a local node with a sufficiently large hard drive could be configured to store all information locally.

A MCK will now be described with more particular reference to the attached drawings.

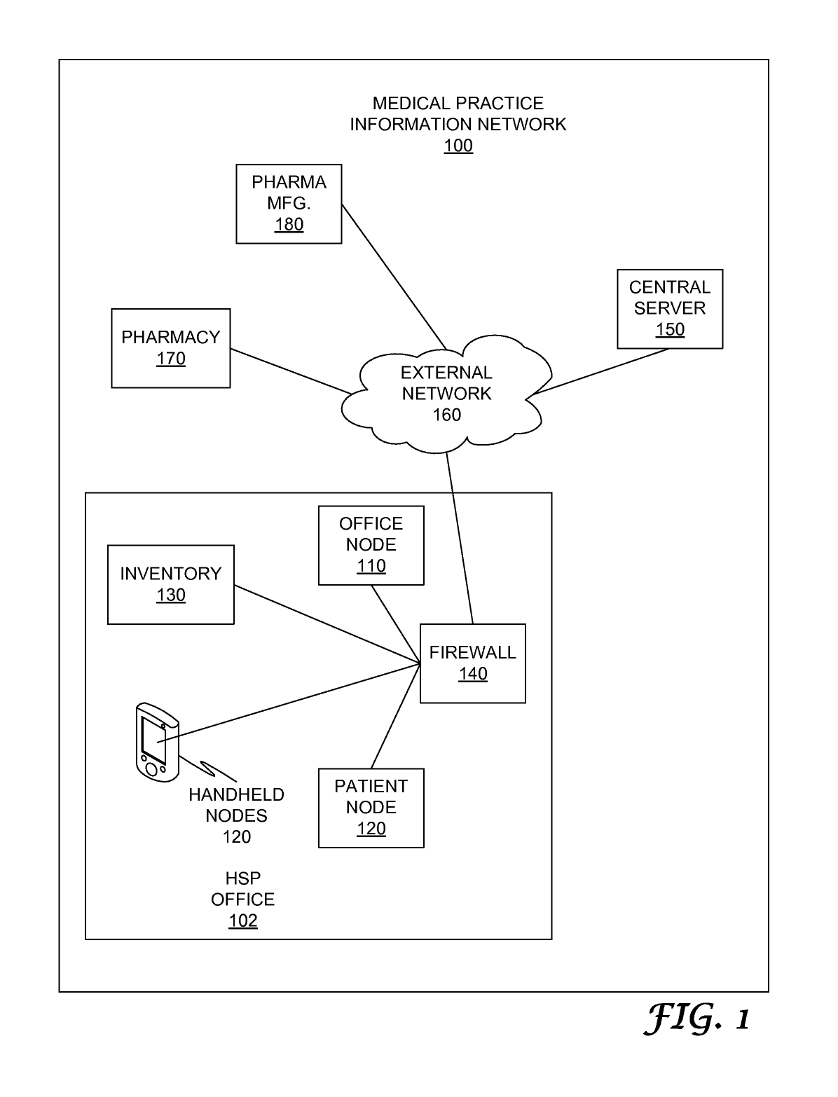

FIG. 1 discloses a block diagram of an MPIN 100. A central server 150 manages all connections between MPIN nodes. In the disclosed embodiment, an external network 160, such as the Internet, is the medium of transfer. But in alternative embodiments, MPIN nodes could connect directly to a central server 150 via a dedicated connection, or via a network other than the Internet. MPIN 100 includes an HSP office 102. HSP office 102 includes a MCK, which in this embodiment includes primarily an office node 110, as well as other nodes providing supporting functions. Office node 110 is the primary node, and is primarily responsible for MCK functions. Patient node 120 is provided in a patient-visible location such as a waiting room, and is useful for performing patient education functions, and may also allow access to a patient portal. In some embodiments, patient node 120 may not include a full hardware platform, but instead may be a thin client or other similar bare-bones hardware arrangement driven by office node 110. Handheld nodes 120 may not be dedicated devices, but instead may be handheld, palmtop, laptop, or other similar portable computing devices configured to interface with office node 110 over a wireless network. Use of handheld nodes 120 can permit the HSP and staff members to access MCK functions without physically being at office node 110.

Each of the nodes at HSP office 102 connects to external network 160 via a firewall, which may also serve as a router. This insures that none of the local nodes are directly connected to an exposed Internet connection.

Also included in HSP office 102 is inventory 130. Inventory 130 is broadly intended to include all drugs, kits, devices, records, and other physical items and data to be tracked by office node 110. In the exemplary embodiment, inventory 130 may include, among other things, a drug sample closet containing a plurality of drug samples, a “crash kit,” medical supplies, medical devices, and EMRs.

A pharmaceutical manufacturer 180 may also interface with central server 150. Pharmaceutical manufacturer 180 can benefit from the rich data available from central server 150. In order to comply with patient privacy regulations, in the exemplary embodiment, central server 150 will never provide personally identifying information to pharmaceutical manufacturer 180. Instead, central server 150 may provide useful information such as demographic data, prescription volumes, and disbursement of samples.

Pharmaceutical manufacturer 180 may also have pharmaceutical representatives who want to visit HSP office 102. In an exemplary embodiment, central server 150 provides two web portals. The first is an HSP-facing web portal, for example like the one currently found at http://www.medgatekeeper.com. The second is a representative-facing portal, such as the one currently found at http://www.drugrepaccess.com. The HSP can logon to medgatekeeper.com and access an online calendar and indicate times when he or she is available to meet with pharmaceutical representatives. The HSP may also indicate the type of medical practice and other useful preferences. For example, an OB/GYN will be targeted for different marketing than a pediatrician. Furthermore, an HSP may also be able to specify how often a particular representative is permitted to visit. For example, the HSP may specify that the representative can only visit once a month. In that case, drugrepaccess.com will not allow the representative to schedule visits more frequently than once a month.

To receive a logon for drugrepaccess.com, the pharmaceutical representative will need to pass a credentialing regimen. Credentialing may include such factors as verifying that the representative is a bona fide employee of a reputable pharmaceutical company, and ensuring that the representative has a history of good behavior on the system. The representative may also be issued an ID badge that has a MCK-readable barcode or other identifier, so that the system can verify that the visiting representative is who he claims to be, and can keep track of visits.

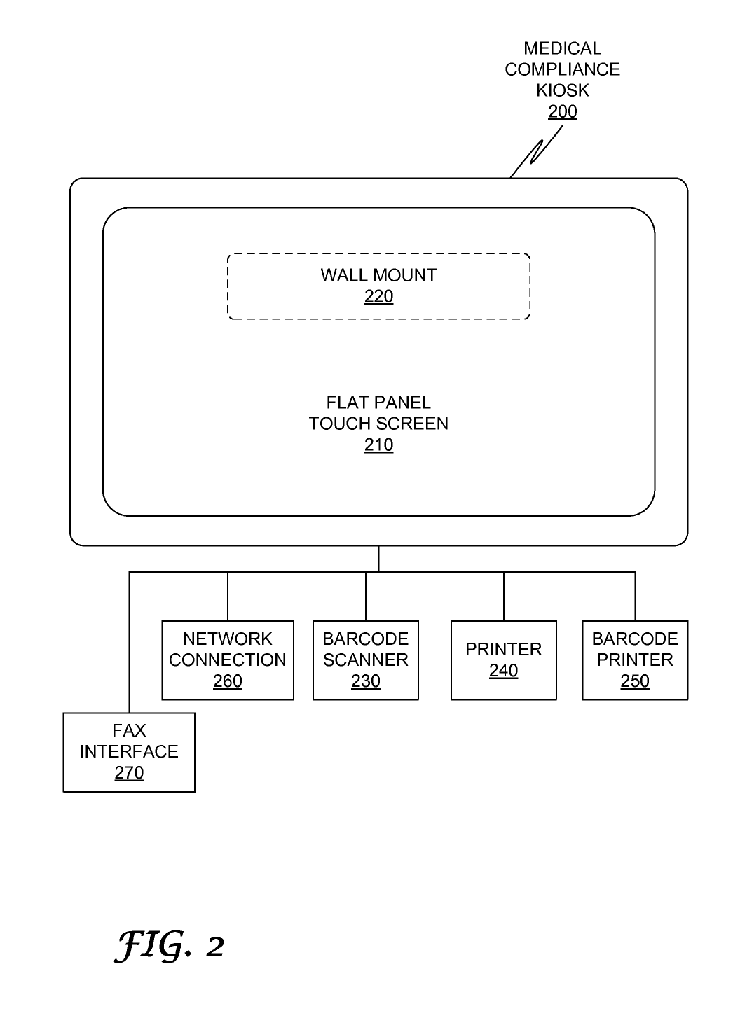

FIG. 2 is a front view of a medical compliance kiosk 200. In the exemplary embodiment of the present specification, some or all of the functions of MCK 200 may be embodied in office node 110 and/or patient node 120. If patient node 120 is a thin client, then all primary functionality will be provided by office node 110. MCK 200 may be provided as a dedicated hardware platform in a sealed casing without any accessible exterior data ports except for optionally a network interface such as an ethernet jack. Providing MCK 200 as a dedicated hardware platform without accessible ports helps to prevent tampering, to maintain control of MCK functionality, and to prevent interference with critical functions, for example by extraneous installed software. In this exemplary embodiment, MCK 200 includes a flat-panel touchscreen 210, which serves as a combined input/output device. When the present specification refers to an input/output device, it is intended to generically refer to any combination of input, output, and/or input/output devices. One purpose for using a flat-panel touchscreen 210 is to eliminate the need for a separate keyboard and/or mouse.

In the exemplary embodiment, a wall mount 220 is provided so that MCK 200 can be located in a highly visible and easily accessible location.

Several peripheral devices may be connected to MCK 200. A barcode printer 250 and barcode scanner 230 are useful for inventory tracking and control functions. Barcode printer 250 may be, for example, a thermal printer configured to print barcode labels on adhesive paper, which can then be affixed to items to be tracked. For example, upon receipt of a new batch of drug samples, a barcode label can be printed for each sample and placed on the samples. Some samples may already have a barcode on the packaging, but the existing barcode may not have sufficient information. For example, all drugs of the same formulation and brand may have an identical barcode, even if they come from different lots and have different expiration dates. An exemplary process for tracking samples with MCK may include inputting the lot number, expiration date, and number of samples into MCK. MCK may then associate a new barcode with these data, and prints enough labels to place on all of the samples. The samples are then placed in the sample closet, and when the sample is taken out, it is scanned, and MCK updates its sample database.

Barcode printer 250 may also be used to prepare barcodes for use with patient records. For example, a barcode can be printed and placed on a folder containing a hard copy of the patient's medical records. When a sample is dispersed, both the sample and the patient's barcode may be scanned. This permits MCK to associate the disbursement of the sample with a specific patient. In the event of a drug recall or warning, MCK can prepare a list of all patients who need to be notified. Because MCK is able to track lot numbers, patients can be appropriately notified even if the recall is targeted to a specific production lot or lots.

To help ease the workload on the HSP, it may be desirable to require the pharmaceutical representative who drops off the samples to register the samples with MCK by checking in the samples. For example, the representative may be required to login to MCK by scanning the barcode on his ID badge and entering a password, and may then be presented with a menu for checking in drug samples. The representative can enter lot numbers and expiration dates for the samples to be dropped off, and print bar code labels for the samples.

A similar procedure may be followed for any other devices, items, or supplies that need to be tracked with MCK. The exemplary embodiment of the specification uses a thermal barcode printer 250 as an exemplary method of performing inventory control, but those having skill in the art will appreciate that other forms of inventory control can be used, and more generically, barcode printer 250 may be referred to as a species of inventory control checkin device. Other inventory control checkin devices may include RFID encoders and/or readers and other similar technologies configured to uniquely identify a specific item or class of items.

Similarly, barcode scanner 230 is provided as an exemplary species of an inventory control checkout device. Other inventory control checkout devices may include RFID readers and other similar technologies.

A printer 240 is also provided. Printer 240 can provide hard copies of patient records and other useful hardcopy information. Printer 240 can also be used when a drug is prescribed or a sample disbursed to print patient education information, such as dosage, instructions, warnings, and interactions.

A fax interface 270 is also provided. In some embodiments, fax interface 270 may not be a fax machine with scanning capabilities, but instead may be a telephone fax modem. Fax interface 270 is useful, for example, when interfacing with a pharmacy that is not configured for direct receipt of electronic prescriptions. In that case, MCK 200 can electronically format a fax page with the prescription information, and also, as necessary, patient insurance information, and deliver the prescription as a fax.

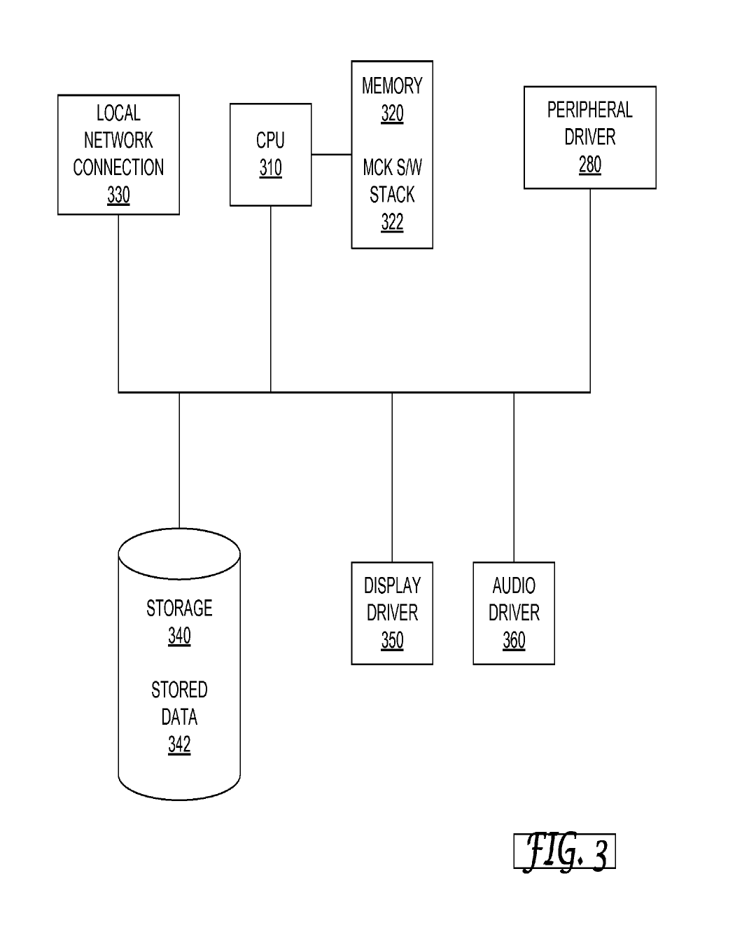

FIG. 3 provides an exemplary embodiment of internal hardware for a MCK 200, such as office node 110. In the exemplary embodiment, MCK 200 is provided as a dedicated hardware device in a sealed casing, with no accessible external data ports except for an optional ethernet jack. In the disclose embodiment, a CPU 310 is connected to a memory 320. Memory 320 may be a low latency, volatile memory medium such as RAM. Stored in memory 320 is MCK software stack 322. MCK software stack 322 contains the operating programs for performing MCK functions. CPU 310 is also connected to a system bus 370, which communicatively couples CPU 310 to other system components. For example, a local network connection 330 may be a hardwired ethernet jack, a WiFi connection, a combination of wired and wireless network access, or other similar technology. CPU 310 is also communicatively coupled to a peripheral driver 380, which allows peripheral devices such as barcode scanner 230, barcode printer 250, printer 240, and fax interface 270 to connect to MCK. A display driver 350 is also communicatively coupled to CPU 310. Display driver 350 provides input and output functions with flat-panel touchscreen 210, or other similar input/output devices. An audio driver 360 may also be communicatively coupled to CPU 310, to provide optional audio features. A nonvolatile storage 340 is communicatively coupled to CPU 310 and has stored thereon stored data 342, including a stored copy of MCK software stack 322. Note that in the exemplary embodiment, EMRs and other sensitive data are not stored locally on nonvolatile storage 340 under normal operating circumstances, but some data, such as the drug sample inventory database, may be stored locally. Under normal operating circumstances, sensitive data are transmitted immediately to central server 150, and will never be saved to nonvolatile storage 340. But if the connection between MCK 200 and central server 150 is lost, local copies of sensitive data may be stored in a local database on nonvolatile storage 340 until the network connection is restored and the local database can be are reconciled with central server 150.

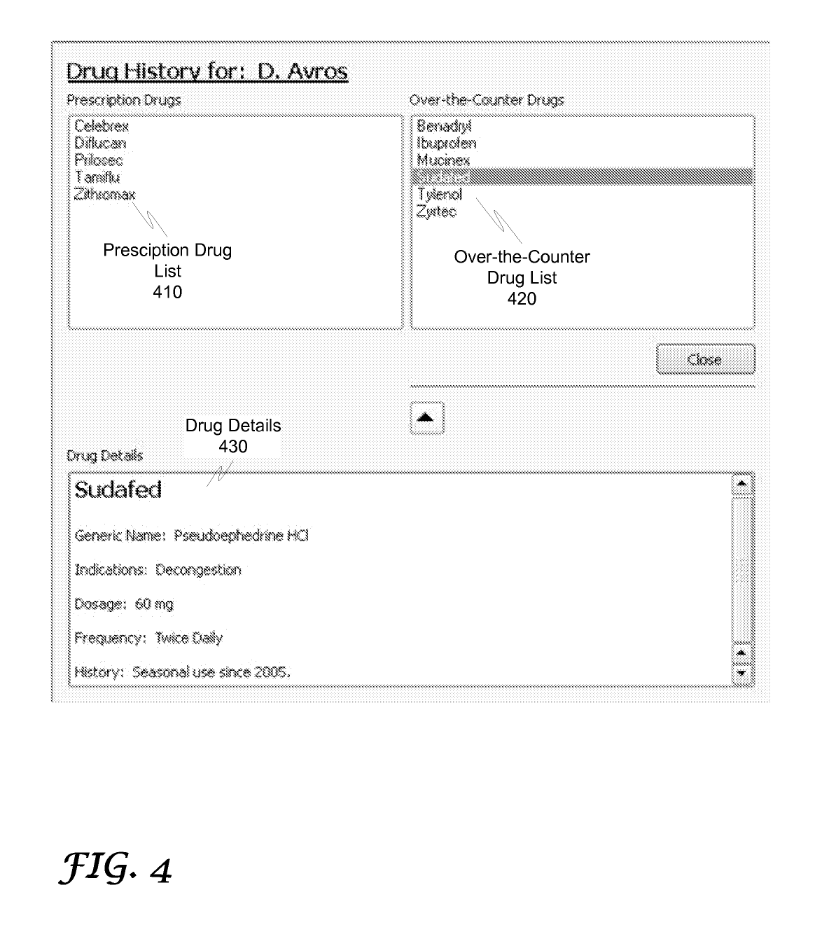

FIGS. 4-7 provide exemplary input screens for a patient portal, as described in more detail below.

### MCK Features and Functions

The following features are provided by way of non-limiting example, in an exemplary embodiment of a MCK.

1. Pharmaceutical Representative Management and Credentialing

A common issue faced by medical practices is the management of pharmaceutical representatives, who persistently want to visit physicians to discuss new drug options and drop off drug samples. MCK provides a system for ensuring that pharmaceutical representatives are properly credentialed, and for allowing the HSP to carefully control the representatives' access to the physician. In particular, MCK interfaces with central server 150, where the physician can log on to an HSP-facing web portal and select blocks of time when the physician is available to meet with pharmaceutical representatives. Properly-credentialed pharmaceutical representatives may then log on to a representative-facing web portal and select desired meeting times.

Credentialing of pharmaceutical representatives may include such items as verifying that the representative is a bona fide employee of a reputable pharmaceutical company, performing background checks on representatives, and maintaining records of the representatives' interactions with the system, including ensuring good behavior.

2. Electronic Medical Records (EMR)

MCK has the ability to provide a consolidated electronic medical record (EMR) for each patient. Because MCK uploads critical data to central server 150 rather than saving it on a local node, a patient who sees several doctors using MCK will have a single consolidated EMR stored on central server 150. Maintenance of all records on central server 150 helps maintain consistency in treatment of a patient, and ensures that all records are maintained in the heavily secured central database. Under normal operating conditions, MCK will not store any records locally, but rather will immediately upload all records to central server 150 and then delete any local copy. In the event of a communication failure between MCK and central server 150, MCK will store data in a local database until communication with the central server 150 is restored. Once communication is restored, all local copies will be deleted.

3. Patient Portal

MCK and/or MPIN have the ability to provide a patient portal for managing patient-oriented information. Patient-oriented information may include EMRs, prescription histories, over-the-counter medications, and other similar patient information.

FIG. 4 is an exemplary user interface screen available on MCK 200 or viewable over the Internet and provided by central server 150, whereby an HSP may see a patient's medication history. A prescription drug list 410 is provided, which lists all or some of the prescriptions given to the patient and entered into a database. An over-the-counter drug list 420 may also be provided so that the HSP can see which over-the-counter medicines the patient is taking. Over-the-counter drugs may be entered directly by the patient by means of an internet or web interface, or the HSP may collect this information and enter it into MCK. There is also shown a box for drug details 430. If the physician wants to see more details about a particular drug, he or she can click on the item and view details in the drug detail screen 430. For example, this patient uses Sudafed twice daily in a 60 mg dosage for decongestion. This patient has been using Sudafed seasonally since 2005. Alternately, if this were a medication that was prescribed for a specific time, then the start and end date of administration of the drug could be listed under the history. For example, if a 10-day course of antibiotics were prescribed, the start date would be the day the antibiotics were prescribed, and the end date would be 10 days later.

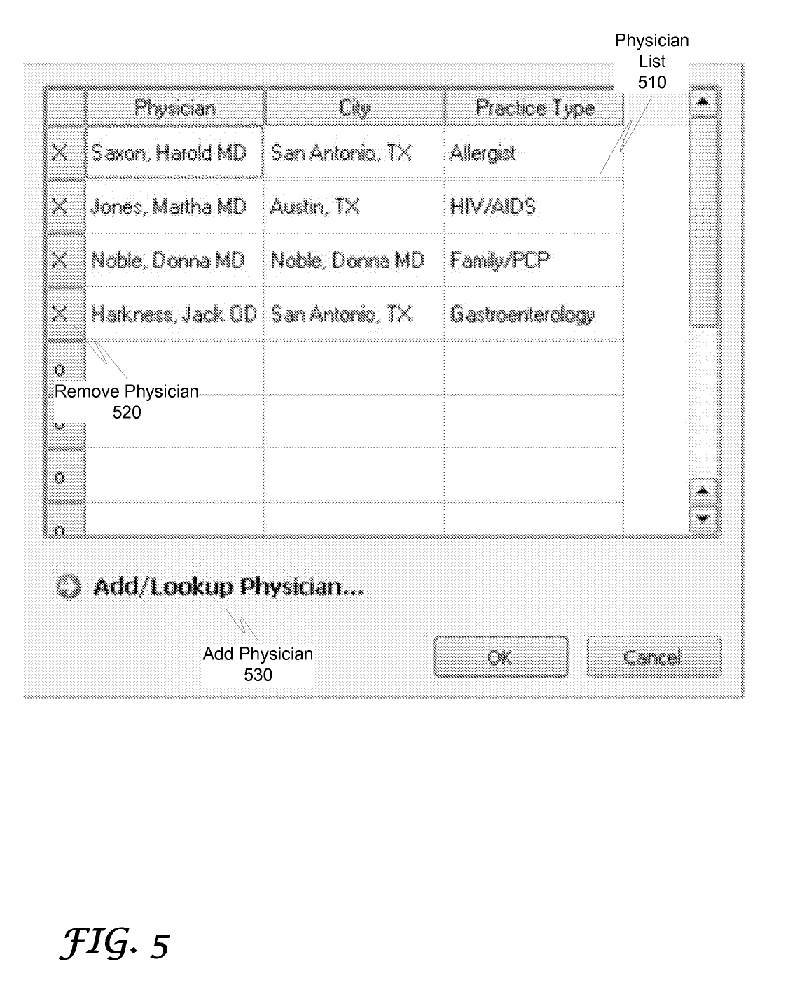

FIG. 5 is an exemplary patient screen, which a patient can view after logging on to the patient portal. This screen may be accessible on MCK, which in this case may be embodied as patient node 120, or over the internet as provided by central server 150. This screen includes a physician list 510, listing the physicians that the patient has chosen to allow access to his personal drug list. A button is also provided to remove physicians 520 so that the patient has the option to revoke a physician's access. Physician list 510 may provide not only the physician's name, but also the city in which he or she practices, and the physician's type of practice. This may help the patient to decide which physicians to give access to, and which drugs to show each physician. There is also a button 530 to add or look up a new physician for the list.

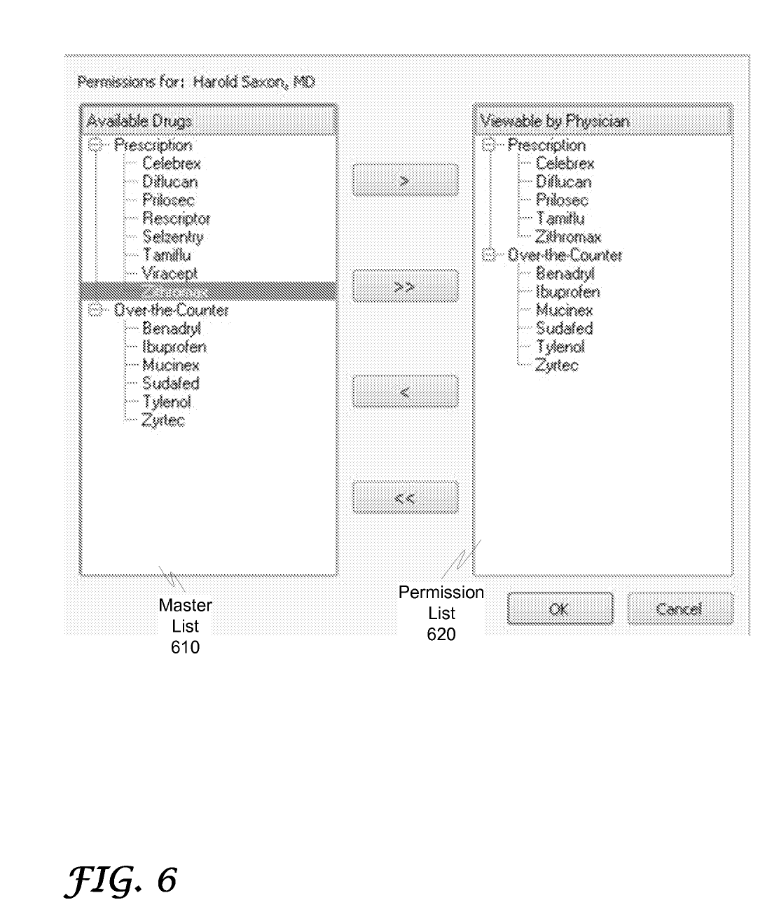

FIG. 6 is an exemplary interface whereby, once the patient has chosen a physician, the patient can fine tune the physician's access to drug information. For example, this patient may be listing drugs that can be seen by his allergist. Included in the database of drugs prescribed to this patient are the drugs Rescriptor, Selzentry, and Viracept. These are drugs that are commonly prescribed for HIV/AIDS. For privacy reasons, the patient may not want his allergist to know that he has HIV/AIDS and may not want to give that physician permission to view the drugs that the patient has taken for HIV/AIDS. So as the patient chooses drugs for his allergist to see, he may exclude Rescriptor, Selzentry, and Viracept, while granting access to the other drugs in the list.

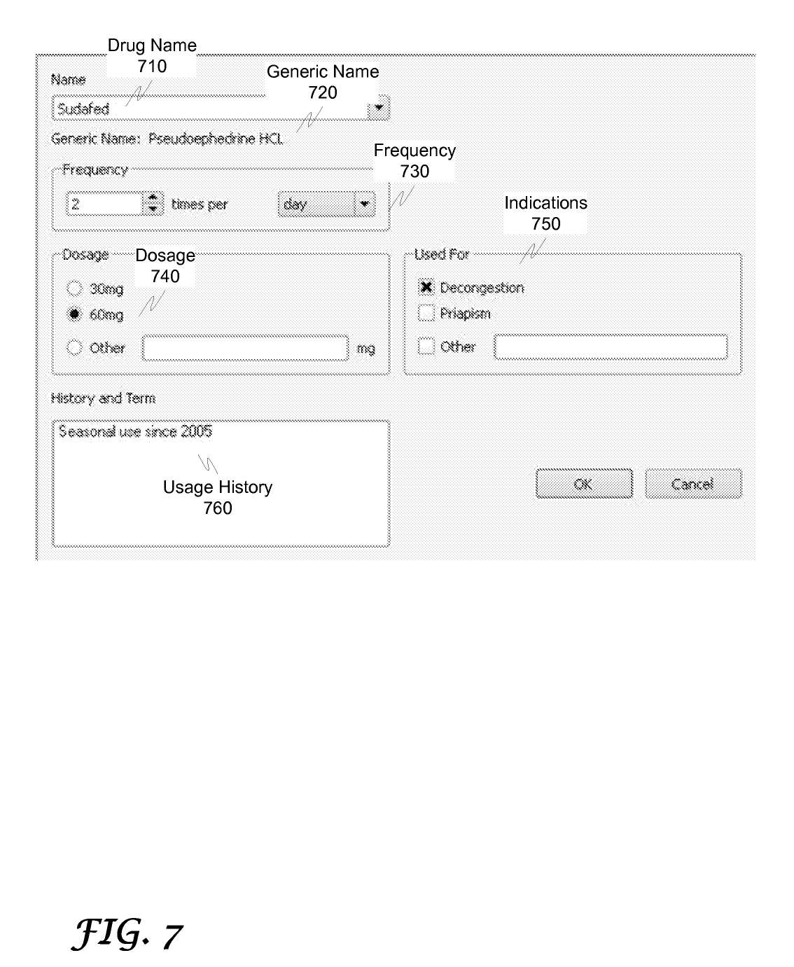

FIG. 7 is an exemplary interface by which a patient or a physician may add a new drug into the patient's database. Different permissions may be associated with certain types of drugs. For example, the patient portal may grant the patient access only to enter over-the-counter drugs, while the HSP may have the ability to enter both over-the-counter and prescription drugs. To ensure integrity of prescription data, the patient may not have the ability to add, delete, or alter any prescription medication records. But the patient would still have the ability to restrict some HSPs from seeing those medications. In an exemplary embodiment, the patient may be able to restrict all physicians except for the prescribing physician or his or her designated successor from seeing any particular drug.

As shown, in this exemplary embodiment, a record is made for the decongestant Sudafed. Because Sudafed is available over-the-counter, this record may be entered by either the patient or a physician. Available fields include drug name 710, generic name 720, frequency of use 730, indications 750, dosage 740, and usage history 760. Some fields may be optional, but providing more information will increase the usefulness of the drug database.

4. Sample Closet Tracking.

Many HSPs maintaint a “sample closet” for storing samples provided by drug companies. Drug samples can be very useful for the physician. For example, when the physician prescribes a medication, the physician can provide the patient with a sample so that the patient has the first several doses of medications available before the prescription is filled. Stocking a physician's sample closet is also useful for pharmaceutical representatives. By providing samples, the pharmaceutical representative reminds the doctor of available drug options, and provides an easy way for the physician to introduce the patient to new drug options.

The samples can also be a source of frustration and difficulty for HSPs. For example, a physician may be fined for having expired drug samples in this sample closet. MCK helps the HSP to maintain an orderly and compliant sample closet.

In one exemplary embodiment, MCK connects to central server 150. On a representative-facing web portal, representative may assign a certain number of vouchers to an HSP. With vouchers, the HSP does not need to maintain and control drug inventories. The HSP can print a voucher for the patient, and the patient can then take voucher to the pharmacy of his choice and receive a free sample of the medication. Providing vouchers rather than physical drugs enables the HSP to focus on medical practice, and worry less about stocking and controlling drug inventories. With vouchers, those tasks can be deferred to the pharmacies, which already have and maintain the infrastructure for managing drug inventories.

In cases where MCK provides vouchers, voucher updates can be provided seamlessly and in near real-time. For example, if a particular physician is running low on vouchers for a particular medication, MCK may send an alert to the designated representative, who can then assign more vouchers to the physician.

To further ease the use of vouchers, and to substantially eliminate paper waste, vouchers can also be transmitted electronically or via facsimile to pharmacies in the name of the patient. Thus, the patient can designate a preferred pharmacy, and when the physician assigns a voucher to the patient, the voucher can be delivered electronically, over the internet or via e-mail, or MCK can automatically format and deliver a facsimile to the pharmacy so that there is no paper for the patient to misplace.

It is conceivable that in the future, vouchers may substantially replace physical drug samples for HSPs. But even with a streamlined process for providing vouchers, some physicians may prefer to have physical samples on hand for at least some kinds of drugs. For example, if the physician wants to be able to provide the patient with an initial dose before a prescription is filled, he may need physical samples on hand.

MCK can also provide substantial benefits in tracking and maintaining physical samples. For tracking samples, it is useful to divide the drug samples into species, each species being identified by a formulation, a lot number, and an expiration date. For example, a physician may have three different drug formulations in his sample closet, identified as drug A, drug B., and drug C, and may have for each of the preceding formulation samples from two different lots with two different expiration dates. In this example, the physician has six species of drugs that need to be tracked. An exemplary process for tracking a plurality of drug samples may include: receiving a plurality of drug samples, the drug samples comprising one or more species, each species being identified by a formulation, a lot code, and an expiration date; providing a barcode for each species of drug sample, for example by printing barcode labels on a thermal printer attached to the MCK, the barcode for each species being distinct from the barcode for the other species; receiving a request to distribute a drug sample to a patient; removing the desired sample from the drug closet; scanning the barcode of the removed drug sample, thus creating a record of the distribution of the sample; and correlating a patient record with the distribution of the sample, for example by scanning a bar code attached to the patient's chart.

Upon request, MCK is able to provide a report of current sample inventories, including the remaining inventory of each formulation, and the expiration date of each remaining sample. MCK may also provide an alert if a particular drug species is nearing expiration. This may alert the physician that the samples nearing expiration should be distributed before newer samples. If MCK determines that there are samples in inventory that have passed their expiration date, MCK can alert the physician that the samples need to be discarded immediately. This helps to prevent the distribution of expired samples, and may prevent the physician from being fined for having expired samples if his sample inventory is inspected. Furthermore, because MCK also provides tracking and credentialing for pharmaceutical representatives, MCK can be aware of which samples come from which representative. Upon detecting that inventory of a particular formulation is low, MCK can alert the relevant pharmaceutical representative, who may then schedule a visit to provide more samples.

The above process also provides advantages with respect to drug recalls or alerts. By associating a patient record with each disbursement of a sample, MCK is able to maintain a database that can be used to identify each patient who has received a particular formulation. Because MCK has the capability of maintaining a complete EMR for each patient, in the event of a drug recall or alert, MCK can automatically generate relevant notices for delivery to affected patients.

MCK can also be used to help alert the physician to potential drug interactions by tracking drugs prescribed by a plurality of physicians.

5. Prescriptions

MCK may be configured to provide simplified prescriptions for patients. An exemplary prior art method of prescribing a drug may include the physician manually writing a prescription on a prescription pad and signing the prescription, delivering the signed prescription to the patient, who then takes the prescription to a pharmacy, where the prescription is filled. In some cases, the prescription may be misplaced, or the pharmacist may misinterpret the written prescription. Furthermore, the pharmacy cannot begin filling the prescription until the patient hand delivers it.

MCK provides a streamlined and more error-resistant method of providing prescriptions. In particular, if the patient's preferred pharmacy is configured to receive electronic prescriptions, MCK can immediately deliver electronic prescription to the patient's preferred pharmacy. This reduces the opportunities for the prescription to be lost, or for the pharmacist to misinterpret the instructions for the prescription. The pharmacy may also begin to fill the prescription immediately, rather than waiting for the patient to hand deliver the prescription. If necessary, the patient's EMR may also include up-to-date insurance information, which can be delivered to the pharmacy.

Some pharmacies may not be configured to receive electronic prescriptions. But the majority of pharmacies are able to receive prescriptions by fax. Some of the advantages of electronic prescriptions can be realized by a providing a direct fax of the prescription. If for example, the physician prescribes a drug, he can enter the relevant information into MCK, and MCK can electronically format a suitable fax page with the relevant prescription data. MCK can then connect to a phone line and dial the pharmacy's fax number and transmit the prescription information as a fax page. With the fax method, there are still fewer opportunities for the prescription to be misplaced or for misinterpretation of handwriting.

Central server 150 may maintain a database of all known interactions for each drug formulation. When a physician prescribe a particular formulation, the known interactions for that formulation can be cross-referenced with other drugs appearing in the patient's EMR. This may help the physician to decide whether to prescribe a particular drug, and may help the physician to provide relevant warnings. For example, if the patient's EMR indicates that the patient took a course of penicillin three years ago to fight a bacterial infection, and penicillin is flagged as a known interaction for the formulation that the physician wants to prescribe, the physician may warn the patient against starting a new penicillin treatment while taking the prescribed drug. Advantageously, with the use of the Patient Portal described in this specification, the physician may have access to this information even if a different doctor prescribed the penicillin.

6. Device Tracking

MCK can also be configured to provide tracking of medical devices. Tracking medical devices may be beneficial for reasons similar to tracking drugs. For example, if there is a recall or alert related to a device such as a prosthetic, artificial joint, or other similar device intended for use with the human body, MCK can track the recipients of each device and provide timely notice of recalls and alerts.

7. “Crash Kits”

Another important issue for medical practices is so-called “crash kits.” Crash kits may include drugs, instruments, and supplies that the physician can use to treat or resuscitate a patient in critical condition. A common issue with crash kits is that for certain types of physicians, it will rarely be used. For example, a primary care physician will not frequently see patients in need of immediate life-saving intervention. But in the rare event that the physician does need to treat such a patient, his crash kit needs to have all of the correct supplies and medications. In particular, because the crash kit is rarely used, there is a serious danger that drugs in the crash kits will be expired when the critical moment comes. There is also the danger that the crash kit has been used and not adequately replenished, so that drugs or supplies are not replaced before the next time the crash kit is needed.

An exemplary method of using MCK to monitor a crash kit may include: providing a barcode for each item in the crash kit; upon encountering a critical care emergency, using supplies and medications from the crash kits as necessary; after stabilizing the patient, scanning the barcode of the each item used; printing or viewing a report of items that need to be replaced; and replacing items in the crash kit. As with sample tracking, MCK can also be configured to provide a warning when drugs in the crash kit are nearing expiration, or to provide an alert and instructions to replace the drugs if they have already expired.

8. Value for Pharmaceutical Manufacturers

As described in this specification, MCK can provide enhanced value and greater efficiency for pharmaceutical manufacturers. For example, central server 150 may record anonymous statistics regarding prescription volumes for particular drugs. To maintain anonymity, the statistics may be stripped of all information personally identifying both the HSP and the patient. In an exemplary embodiment, statistics are reported with respect to a zip code or other regional designation. This may help the pharmaceutical manufacturers better focus marketing resources.

Manufacturers may also be permitted to purchase advertising time that will run in lieu of a screensaver on individual MCK nodes. For example, the manufacturer may prepare a thirty second electronic primary detail equivalent (EPDE). The EPDE can be targeted toward the physician and include highly relevant information designed to educate the physician on the indications, benefits, risks, and side effects of the advertised drug formulation.

Purchase of an EPDE may be managed by logging on to a website provided by central server 150 and uploading the desired content. In the exemplary embodiment, an EPDE is purchased in a block of thirty second segments. When purchasing an EPDE block, the manufacturer may designate zip codes or other regional designations, as well as types of practices to receive EPDE. This permits the manufacturer to specifically target the most productive regions. Central server 150 will then push EPDE packages to each individual physician's office.

When a MCK node is sitting idle, it will display an EPDE in lieu of a screensaver. By default, the display will be muted, but a single click unmute button may be provided so that the HSP can hear the accompanying audio on demand.

9. Patient Education

Patient node 120 may be located in a waiting room or other location where it is visible to patients. In some embodiments, patient node 120 may provide limited functionality, and therefore may be a thin client to minimize cost. Patient node 120 may provide patient-oriented advertisements similar to the EPDE provided to the physician. As with EPDE, patient-oriented advertisements may be purchased via a web interface on central server 150 according to zip code or other regional designation. Patient node 120 may also provide general patient education material, such as information about the medical practice, the physician or physicians practicing there, services provided, as well as display the names of patients currently being called back to see a doctor and other real-time status updates. In some embodiments, patients in the waiting room may be allowed to log on to the Patient Portal at patient node 120.

While the subject of this specification has been described in connection with one or more exemplary embodiments, it is not intended to limit the claims to the particular forms set forth. On the contrary, the appended claims are intended to cover such alternatives, modifications and equivalents as may be included within their spirit and scope.

What is claimed is:

1. A medical compliance kiosk comprising:
a dedicated hardware platform comprising
a processor;
a combined input/output device communicatively coupled to the processor and provided to receive input from a user and provide output to the user;
a network connection communicatively coupled to the processor and configured to communicatively couple the processor to a local network and to an external network, the external network being communicatively coupled to a central server;
a peripheral driver communicatively coupled to the processor and configured to communicatively couple the processor to an inventory control checkin device and an inventory control checkout device.
a sealed casing providing no more than one accessible data port;
a tangible memory medium;
wherein the tangible memory medium has stored thereon software instructions that, when executed, instruct the processor to:
provide a patient portal wherein a patient may interact with the medical compliance kiosk and manage permissions for a plurality of health service providers (HSPs) to view the patient's drug history;
create electronic medical record (EMR) data and transmit the EMR data to the central server;
manage an inventory of drug samples, including checking in of new samples received, checking out of samples disbursed, tracking of sample recipients, and notification of drug sample expirations;
pharmaceutical representative management, including credentialing of representatives and scheduling of physician time for representatives;
medical device management, including tracking of medical device inventories and recipients of medical devices;
crash kit management, including tracking of supplies used from a crash kit and expiration of drugs in a crash kit;
patient education, including pharmaceutical advertisements, and prescription information; and
electronic prescription management.

2. The medical compliance kiosk of claim 1 wherein the accessible data port is an Ethernet port.

3. The medical compliance kiosk of claim 1 where the local network includes a firewall at a sole connection point to the external network.

4. The medical compliance kiosk of claim 1 wherein EMRs are stored on the medical compliance kiosk only in the event of a communication failure between the medical compliance kiosk and the central server.

5. The medical compliance kiosk of claim 1 wherein the patient portal is further configured to:
provide only the HSP the ability to enter information on prescription drugs;
provide only patients or the HSP the ability to enter information on over-the-counter drugs.

6. The medical compliance kiosk of claim 1 wherein pharmaceutical representative management further comprises:
providing a first internet-accessible portal accessible to a representative only after meeting credentialing requirements;
providing a second internet-accessible portal for the HSP;
providing on the second portal an interactive calendar wherein the HSP can indicate times available for meeting with representatives; and
providing on the first portal an interactive calendar wherein representatives can view available times for meeting with the HSP and schedule a meeting.

7. The medical compliance kiosk of claim 6 wherein the credentialing requirements include verifying that the representative is a bona fide employee of a reputable pharmaceutical manufacturer.

8. The medical compliance kiosk of claim 1 wherein the instructions further comprise instructions to:
provide an electronic primary data equivalent display on a repeating loop when the medical compliance kiosk is inactive.

9. The medical compliance kiosk of claim 1 wherein the medical compliance kiosk is communicatively coupled to a bar code scanner and a bar code printer; and wherein checking in an item comprises printing a bar code on the bar code scanner, and checking out an item comprises scanning the bar code.

10. The medical compliance kiosk of claim 9 wherein managing the inventory of drug samples further comprises:
verifying a pharmaceutical representative's identity;
granting the representative access to check in drug samples, wherein checking in drug samples comprises
receiving from the representative at least one drug species to be placed in inventory, the species including a formulation, a lot code, and an expiration date;
receiving from the representative a quantity of drugs of the species;
printing a barcode label for each drug sample; and
updating a drug sample database by adding the drug samples.

11. The medical compliance kiosk of claim 9 wherein checking out a drug sample further comprises scanning a bar code associated with a patient and updating an EMR for the patient by adding the checked out drug sample.
